(19) United States
(12) Patent Application Publication
(10) Pub. No.: US 2008/0119665 A1
(43) Pub. Date: May 22, 2008

(54) Process for the Synthesis of Zirconium Carboxylates

(76) Inventors: Peter Fritz, Unterhaching (DE); Heinz Bolt, Wolfratshausen (DE); Karl-Heinz Scherschlicht, Mossling (DE); Fuad Mosa, Riyadh (SA); Talal K. Ali, Riyadh (SA); Sultan Al-Otaibi, Riyadh (SA)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 11/662,150

(22) PCT Filed: Aug. 10, 2005
(86) PCT No.: PCT/EP05/08671

§ 371 Date: Nov. 14, 2007

(30) Foreign Application Priority Data
Sep. 7, 2004 (DE) 102004043271.6

(51) Int. Cl. C07C 51/41

(52) U.S. Cl. 556/55

(57) ABSTRACT

A process for the synthesis of zirconium tetra carboxylates is disclosed. To overcome the disadvantageous characteristics of the currently used production process, which include highly corrosive starting substances and products, and complex removal of HCl, ZrCI₄ is suspended in an apolar solvent and reacted with the anhydride of an organic acid. The sole products of this reaction are slightly corrosive Zr tetra carboxylate and an organic acid chloride, which is a valuable basic chemical in the chemicals and pharmaceuticals industry. The reactors required for the synthesis need not necessarily consist of glass and/or enamel, but may instead be manufactured from stainless steel, which is cheaper. Apparatuses for the removal and treatment of hydrochloric acid are not necessary.

The invention relates to a novel process for the synthesis of zirconium carboxylates.

Zirconium carboxylates are used as a part of catalyst systems for the production of linear α-olefins (LAO). The LAO synthesis reaction here proceeds in an organic solvent.

Linear α-olefins are compounds which are, for example, widely used and required in large quantities as comonomers for modifying the properties of polyolefins or as a starting material for the production of plasticisers, surface-active substances and the like. Several hundred tons of zirconium carboxylate solution are required for the annual output from a large-scale industrial LAO plant. The production of this large quantity of catalyst material typically proceeds in reactors with a capacity of approx. 15 m³.

Zirconium chloride (ZrCl₄) is already suitable as a catalyst for LAO synthesis (U.S. Pat. Nos. 4,486,615 and 4,783,573). However, due to the low solubility thereof in organic solvents, the more readily soluble and thus substantially more active zirconium carboxylates are in practice used instead of ZrCl₄ (USSR Author's Certificate 1042710). Zirconium carboxylates are currently produced in a process as stated in Linde patent DE4338416 C1, in which ZrCl₄ is reacted in an organic solvent with isobutyric acid (HX) to yield zirconium carboxylate (ZrX₄) and hydrochloric acid in accordance with the equation

ZrCl₄+4HX→ZrX₄+4HCl

Both the starting substance isobutyric acid and the product hydrochloric acid are extremely corrosive. It is accordingly not possible to use a stainless steel reactor for the synthesis, as the alloy constituents, such as iron, chromium, nickel or titanium, contained in the steel are leached out and, as contaminants, severely impair the catalytic action of ZrX₄. For this reason, the reactor and the other plant components, such as stirrers and pipework, consist of glass and/or enamel. While these materials do indeed withstand the corrosive attack from the substances involved in the synthesis, they result in elevated capital costs for the synthesis plant. While a production plant with a reactor (approx. 15 m³) made from glass/enamel together with apparatuses for the removal of the hydrochloric acid from the solution and for treatment of the exhaust gases containing hydrochloric acid entails capital investment of several million Euros, an identically sized plant made from stainless steel and without the apparatuses for removal and treatment of hydrochloric acid may be built for a fraction of this cost.

Apart from elevated plant costs, a further disadvantage of the above-described process is that even a few ppm of contamination with HCl considerably impair the catalytic activity of the zirconium carboxylates. Although a proportion of the hydrochloric acid is already drawn off in the gas phase during synthesis, the hydrochloric acid remaining in the solution must be removed in a subsequent process step, which lasts several days, by stripping with nitrogen. The long duration of this process step considerably limits the productivity of the process. Moreover, the consumption of large quantities of nitrogen entails high operating costs.

The object of the present invention is accordingly to provide a process of the above-stated type which enables the large-scale industrial synthesis of zirconium carboxylates without involving hydrochloric acid.

This object is achieved according to the invention in that ZrCl₄ suspended in an organic solvent is reacted with the anhydride of an organic acid and, in particular not hydrochloric acid, but instead zirconium carboxylate and an acid chloride are produced in this reaction.

In accordance with the process of the invention, toluene, benzene, xylene, hexane or chlorobenzene are preferably used as the apolar solvent for Zr carboxylate synthesis.

The chemical reaction here proceeds in accordance with the equation

ZrCl₄+4R—CO—O—CO—R′→Zr(O—CO—R)₄+Cl—CO—R′

in which the abbreviations R and R′ denote methyl, ethyl, propyl, isopropyl, butyl, phenyl, hexyl or benzyl, wherein R′ is identical to R or R′ alternatively designates methyl.

The process yields as intermediate product a solution of Zr carboxylate and the acid chloride in the solvent used. The acid chloride secondary product may straightforwardly be removed from the solution by distillation. It is a valuable basic chemical in the chemicals and pharmaceuticals industry and may profitably be sold as such.

1. A process for the synthesis of zirconium tetra carboxylate and organic acid chloride, comprising suspending ZrCl₄ in an apolar organic solvent and reacting it with the anhydride of an organic acid.

2. The process of claim 1, wherein the zirconium tetra carboxylate is synthesized in accordance with the reaction equation
ZrCl₄+4R—CO—O—CO—R′→Zr(O—CO—R)₄+4Cl—CO—R′,
wherein R is methyl, ethyl, propyl, isopropyl, butyl, phenyl, hexyl or benzyl, and R′ is either identical to R or is methyl.

3. (canceled)

4. The process according to claim 1 wherein the organic apolar solvent comprises toluene, benzene, xylene, hexane or chlorobenzene.

5. (canceled)

6. The process according to claim 2, wherein the organic apolar solvent comprises toluene, benzene, xylene, hexane or chlorobenzene.
